(19) United States
(12) Patent Application Publication
(10) Pub. No.: US 2024/0008982 A1
(43) Pub. Date: Jan. 11, 2024

(54) SYSTEM AND METHOD FOR THE DELIVERY OF AN ANNULOPLASTY DEVICE

(71) Applicant: Vestlandets Innovasjonsselskap AS (VIS), Bergen (NO)

(72) Inventors: Vegard TUSETH, Bergen (NO); Philip Jon HAARSTAD, Chanhassen, MN (US); Jacob JAEGER, White Bear Lake, MN (US); Lucas HARDER, Minneapolis, MN (US); Matt KEILLOR, Saint-Sébastien-sur-Loire (FR)

(21) Appl. No.: 17/862,177

(22) Filed: Jul. 11, 2022

(51) Int. Cl. A61F 2/24; A61B 17/00

(52) U.S. Cl. CPC A61F 2/2445; A61B 17/00234; A61F 2/2466

(57) ABSTRACT

The present invention relates to a system for the delivery of an annuloplasty device for restoring a heart valve annulus from a dysfunctional configuration to a working configuration, wherein the annuloplasty device is configured to expand from a pre-set configuration corresponding to the working configuration of the valve annulus, the system comprising a delivery device configured to be detachably coupled to the annuloplasty device, wherein the delivery device comprises a structure configured to expand from a first configuration to a second configuration, wherein the dimensions of the second configuration are substantially the same or greater than the dimensions of the pre-set configuration of the annuloplasty device.

The present invention relates to systems and methods for the delivery of a medical device, such as an implant. More particularly the present invention relates to systems and methods for the delivery of an annuloplasty device.

### BACKGROUND OF THE INVENTION

Annuloplasty is a procedure for the repair of a heart valve, by tightening or strengthening the annulus around a valve. Annuloplasty procedures are often used to treat heart valve regurgitation. By way of example, the mitral valve regulates blood flow from the left atrium to the left ventricle, and prevents blood from flowing back into the left atrium from the left ventricle. Mitral valve regurgitation is a condition which occurs when blood flows backwards into the left atrium across a so-called “leaky heart valve”. This backflow is caused by a dysfunctional heart valve, which may be dysfunctional through injury, malformation and/or disease.

If the condition is minor, no or little treatment is required. However, in some cases, the blood backflow places a strain on the heart and causes it to work harder to compensate for the leaked volumes. A conventional and effective surgical method for treating this condition is with the use of an annuloplasty band or ring, which reduces the size of the annulus around the leaky valve to restore normal working function.

In recent years, alternatives procedures, in particular using transcatheter technologies, have been developed to treat patients for whom surgical intervention is unsuitable. Transcatheter or percutaneous procedures are minimally invasive, and hence minimally traumatic.

A major challenge in transcatheter procedures is accessibility, including the delivery of medical tools and devices to the target site, and their precise and safe manipulation at the target site. Despite considerable advances in this field, it remains difficult to implant miniaturised devices in areas hindered by the patient's anatomy. Moreover, the medical practitioner must take into account the specific anatomical landscape such as shape, dimensions, damage and condition) of the target area for a successful implantation.

Whilst in surgical procedures, an annuloplasty device of suitable shape and size can be directly manipulated, positioned and attached to the dysfunctional annulus, transcatheter implantation requires imaging equipment to visualise the device delivery path and the target site. There is also added complexity due to the accessibility of the target area, lack of direct visualisation and reduced size of the transcatheter tools and devices.

In transcatheter procedures, the annuloplasty device is delivered (and the medical tools manipulated) through a catheter, which conventionally travels through the patient's circulatory system. Due to the size restrictions, it is therefore difficult, if not impossible, to deliver an annuloplasty device with a fixed configuration. The fixed configuration would be the configuration of the device required to restore the annulus to a working configuration, and the lumen of the delivery catheter would need to be sufficiently large not only to accommodate this final configuration, but also to provide unhindered space for the surgeons to manipulate the tools and devices. Such a large catheter cannot travel through the patient's circulatory system. As a result, transcatheter annuloplasty devices are contractible or collapsible to enable delivery through a catheter.

Most established transcatheter annuloplasty procedures involve the use of a flexible or semi-flexible band or ring. Once at the target area, the band or ring is positioned by the surgeon, sutured, screwed or otherwise anchored to the degenerated annulus to form the desired final working configuration, and subsequently tightened to narrow the annulus.

Thus, it is a requirement for transcatheter annuloplasty devices to be collapsible in order to be delivered through a catheter, and a consequence of this requirement is that the surgeon must reconstruct in situ the working configuration of the annuloplasty device.

The heart valve anatomy is complex in itself, and repairing a heart valve with the currently available techniques is equally so. The development of new annuloplasty devices and of corresponding delivery tools, is costly, and medical practitioners are required to undertake extensive and intensive training specific to each device-type before being able to fit them into patients. The process of establishing each new device in current medical practice is therefore highly resource-intensive.

Moreover, even with sophisticated devices and tools, intensive training and expert surgical intervention, there are so many variables at play that the outcome of the procedure is unpredictable. It is difficult to predict whether the band has been suitably positioned and anchored so that regurgitation may be significantly minimised, until the band has been tightened. At this stage, the device has already been sutured to the annulus, and can only be retrieved through surgery, if at all. It is also difficult to predict if the restoration of the valve is durable. In addition, repeated attempts at successful implantation and trial and error is damaging and traumatic to the patient's anatomy. Despite all efforts, the band may still fail to successfully restore the valve.

It is an object of this invention to mitigate problems such as those described above and to provide an improved alternative to existing products.

In particular, there is a need for a simpler, more standardised, less resource-intensive and less traumatic system and method for repairing leaky heart valves.

There is further a need for a system and method for the easy manipulation, efficient delivery and accurate positioning of an annuloplasty device. There is also the need for a system and method which enable the repositioning of an annuloplasty device wherever required.

### SUMMARY OF INVENTION

According to a first aspect of the invention, there is provided a system for the delivery of an annuloplasty device for restoring a heart valve annulus from a dysfunctional configuration to a working configuration. The annuloplasty device is configured to expand from a pre-set configuration corresponding to the working configuration of the valve annulus. The system comprises a delivery device configured to be detachably coupled to the annuloplasty device. The delivery device comprises a structure configured to expand from a first configuration to a second configuration, wherein the dimensions of the second configuration are the substantially the same or greater than the dimensions of the pre-set configuration of the annuloplasty device.

The delivery system is configured and arranged to be expanded so that the annuloplasty device can be positioned adjacent the relevant anchoring locations on and around the annulus. Once, the annuloplasty device is suitable positioned, it may be released so as to revert to its pre-set configuration, which restores the annulus into its working configuration. The annuloplasty device may also be configured and arranged so as to be folded to fit into a delivery sheath. The system according to the present invention has been specifically developed and adapted to the delivery of such an annuloplasty device. In particular, the delivery system configured and arranged so as to grab the annuloplasty device, to expand it beyond its pre-set configuration for positioning, to release it thereby allowing the annuloplasty device to revert to its pre-set configuration and to anchor to the annulus.

The expandable structure of the delivery device may comprise a shaft and one or more arms. The arm(s) may be movably connected to the shaft. The arm(s) may for example be pivotably connected to the shaft. The arm(s) may have a proximal end and a distal end, and the proximal end may be connected to the shaft. Preferably, the proximal end of the arm is fixedly connected to the shaft (i.e. at one connection point on the shaft) so that the arms is movable or pivotable about the connection between the proximal end of the arm and the shaft.

The arms may be configured to fold radially inwards into the first configuration. In other words, the arms may be folded inwards towards the shaft (e.g. radially inwards relative to the longitudinal axis of the shaft), i.e. the distance between the distal ends of the arms and the shaft is decreased. The first configuration may be referred to as a “folded configuration”, which allows the delivery system to fit within a delivery sheath, for example for the delivery, release and/or retrieval of the delivery system.

The arms may be configured to expand radially outwards into the second configuration. In other words, the arms may be expanded away from the shaft (e.g. radially outwards relative to the longitudinal axis of the shaft), i.e. the distance between the distal ends of the arms and the shaft is increased. The second configuration may be referred to as an “expanded configuration”, which allows the delivery system to be expanded beyond it pre-set configuration. Thus, the annuloplasty device may be manipulated, positioned and/or repositioned prior to release.

The delivery system may comprise an actuator for controlling the expansion of the expandable structure. The actuator enables the adjustment and control of the degree of expansion of the expandable structure. Where the expandable structure comprises a plurality of arms, the actuator may control the expansion of an individual arm and/or of one or more sets of arms, or of all the arms simultaneously. The actuator may be configured and arranged so that the distal expansion of the arms (at the patient's end) is controlled from the proximal side (user side) of the delivery system.

The actuator may comprise an actuating rod. The rod may be slidable relative to the longitudinal axis of the shaft. The actuator may comprise one or more stretchers.

The stretchers may be coupled to the arms and the actuating rod. The stretcher(s) may be movably coupled to the arm(s) and/or the actuating rod. For example, the stretcher(s) may be pivotably coupled to the arm(s) and/or to the actuating rod.

The stretcher may have a proximal end and a distal end. The distal end of the stretcher may be coupled to the actuating rod, and the proximal end of the stretcher may be coupled to the arm(s). Preferably, the proximal end of the arm is fixedly connected to the arm(s) and/or to the actuating rod (i.e. at one connection point) so that the stretcher is movable or pivotable about the connection between the proximal and distal end of the stretcher, and the arm(s) or to the actuating rod, respectively.

The actuating rod may have a proximal end and a distal end. The stretchers may be coupled to the distal end, or adjacent the distal end of, the actuating rod.

Each arm comprises a proximal end, a distal end and an intermediate portion extending therebetween. In some embodiments, the stretchers are coupled to the intermediate portions of the arms. In some embodiments, the stretchers are coupled to adjacent the proximal ends of, the arms.

A stretcher may extend between the actuating rod and one arm, thereby allowing the individual expansion or folding of said arm. A stretcher may extend between the actuating rod and two or more arms, thereby allowing the simultaneous expansion or folding of said two or more arms.

In some embodiments, the shaft comprises one or more lumens. The actuating rod may be housed in the/a lumen of the shaft. The actuating rod may be configured and arranged to slide within the/a lumen of the shaft. The actuating rod may be configured and arranged to slide beyond the distal end of the shaft, thereby radially expanding the arms.

In some embodiments, the actuator may comprise an actuating sleeve. The actuating sleeve may be slidable relative to the longitudinal axis of the shaft. The actuator may comprise one or more stretchers coupled to the arms and the actuating sleeve.

The stretcher(s) may be movably coupled to the arm(s) and/or the actuating sleeve. For example, the stretcher(s) may be pivotably coupled to the arm(s) and/or to the actuating sleeve.

The stretcher may have a proximal end and a distal end. The proximal end of the stretcher may be coupled to the actuating sleeve, and the distal end of the stretcher may be coupled to the arm(s). Preferably, the proximal end of the arm is fixedly connected to the arm(s) and/or to the actuating sleeve (i.e. at one connection point) so that the stretcher is movable or pivotable about the connection between the distal or proximal end of the stretcher, and the arm(s) or to the actuating sleeve, respectively.

The actuating sleeve may have a proximal end and a distal end. The stretchers may be coupled to the distal end, or adjacent the distal end of, the actuating sleeve.

Each arm comprises a proximal end, a distal end and an intermediate portion extending therebetween. In some embodiments, the stretchers are coupled to the intermediate portions of the arms. In some embodiments, the stretchers are coupled adjacent the proximal ends of, the arms.

A stretcher may extend between the actuating sleeve and one arm, thereby allowing the individual expansion or folding of said arm. A stretcher may extend between the actuating sleeve and two or more arms, thereby allowing the simultaneous expansion or folding of said two or more arms.

The actuating sleeve may comprise one or more lumens. The shaft may be housed in the/a lumen of the actuating sleeve.

The shaft may be configured and arranged to slide within the/a lumen of the actuating sleeve. The shaft may be configured and arranged to slide beyond the distal end of the actuating sleeve, thereby radially expanding the arms.

The delivery device may comprise one or more connectors for detachably coupling the expandable structure to the annuloplasty device. The connector may comprise a detachable loop connector or hook connector.

The loop may comprise one or more severable sections. The loop may surround a portion of the annuloplasty device, thereby securing the annuloplasty device. The loop may be severed (for example by breaking, rupturing, dissolving or by other suitable manner) to release the annuloplasty device.

The hook connector may be made of a shape-memory material, so that in its pre-set configuration the connector forms a hook. In its pre-set configuration, the hook may partially surround a portion of the annuloplasty device to secure the annuloplasty device. The hook may be deformed to release the annuloplasty device.

In some embodiments, the connector may comprise one or more ribs configured and arranged to receive a connector control line. The ribs may comprise a distal end coupled to the distal end, or adjacent the distal end, of an arm.

In some embodiments, one or more arms are configured and arranged to receive a connector control line.

The rib and/or arm may comprise a connector outlet. The connector outlet may be located at the distal end of a rib and/or arm, so as to allow the loop connector or hook connector to extend beyond the distal end of the rib and/or arm. The connector outlet may be pivotably coupled to said rib and/or arm.

The connector outlet may be advantageously configured to protect the patient's surrounding tissues from injury, by providing an atraumatic surface.

The connector outlet may be pivotably, resiliently or otherwise movably coupled to the rib and/or arm, relative to said rib and/or arm, to provide a surface aligned with the patient's surrounding anatomy. The surface may be substantially flat or curved, and/or comprise or consist of a flexible or soft material which will not injure the patient.

In some embodiments, the delivery system further comprises the annuloplasty device.

According to a second aspect of the invention, there is provided a method for restoring a heart valve annulus, comprising the step of delivering an annuloplasty device for restoring a heart valve annulus from a dysfunctional configuration to a working configuration, wherein the annuloplasty device is configured to expand from a pre-set configuration corresponding to the working configuration of the valve annulus, the system comprising a delivery device configured to be detachably coupled to the annuloplasty device, using a delivery system according to the present disclosure.

The method may comprise the step of coupling the annuloplasty device to the expandable structure of the delivery device.

The method may comprise the step of inserting the delivery system into a delivery sheath, wherein the expandable structure is in a folded configuration.

The method may comprise the steps of expanding the expandable structure into the second configuration.

The method may comprise the step of releasing the annuloplasty device into its pre-set configuration.

In some embodiments, the heart valve is the tricuspid valve, and the annulus is the anterior annulus, the posterior annulus and/or the septal annulus.

### ACCOMPANYING FIGURES

The details of one or more implementations are set forth, by way of example only, in the accompanying drawings and the description below. Other features, aspects, and advantages of the disclosure will become apparent from the description, the drawings, and the claims, in which:

FIG. 1A is a schematic representation of an annuloplasty device (“band-type” or “ring-type” annuloplasty device), in its pre-set configuration;

FIG. 1B is a schematic representation of the annuloplasty device of FIG. 1A in an expanded configuration;

FIG. 1C is a schematic representation of the annuloplasty device of FIG. 1A in a compressed configuration;

FIG. 1D is a schematic representation of the annuloplasty device of FIG. 1A illustrating anchoring means;

FIG. 2A is a schematic representation of an annuloplasty device, in its pre-set configuration;

FIG. 2B is a schematic representation of the annuloplasty device of FIG. 2A an expanded configuration;

FIG. 2C is a schematic representation of the annuloplasty device of FIG. 1A a compressed configuration;

FIG. 2D is a schematic representation of the annuloplasty device of FIG. 2A illustrating anchoring means;

FIG. 3A is a schematic representation of an annuloplasty device, in its pre-set configuration;

FIG. 3B is a schematic representation of the annuloplasty device of FIG. 3A a compressed configuration;

FIG. 4A is a schematic top representation of a working mitral valve;

FIG. 4B is a schematic representation of the three-dimensional shape of a working mitral valve;

FIG. 5 is a schematic top representation of a working tricuspid valve;

FIGS. 6A to 6J are schematic top representations of annuloplasty device shapes;

FIG. 7A to 7C are schematic representations of an annuloplasty device (“crown-type” annuloplasty device), its delivery configuration, in its expanded configuration and in its pre-set configuration, respectively;

FIGS. 8A and 8B shows an annuloplasty device with anchoring means extending inwards;

FIGS. 9A and 9B are schematic illustrations of an annuloplasty device “bridge-type” annuloplasty device);

FIG. 9C is a schematic representation of anchoring means for use in an annuloplasty device according to the present invention;

FIGS. 10A, 10B, 10C are schematic representations of a delivery system according to the present invention;

FIGS. 11A, 11B, 11C are schematic representations of a delivery system according to the present invention;

FIGS. 12A and 12B are schematic representations of a delivery system according to the present invention;

FIG. 13 is a schematic representation of part of a delivery device 100, coupled to an annuloplasty device 20 of the “crown-type”; and

FIGS. 14 and 15 are schematic representations of an annuloplasty device (“crown-type” annuloplasty device), its expanded configuration and in its pre-set configuration, respectively.

The embodiments described herein are provided as exemplary and non-limiting embodiments of the present invention.

### DETAILED DESCRIPTION

The present disclosure concerns a system for the delivery of an annuloplasty device for restoring a heart valve annulus from a dysfunctional configuration to a working configuration, wherein the annuloplasty device is configured to expand from a pre-set configuration corresponding to the working configuration of the valve annulus. The delivery system may be referred to, interchangeably, as an annuloplasty system. The annuloplasty device will be described first, and the delivery system and method will subsequently be described by reference to the annuloplasty device.

### Annuloplasty Device

The system of the present disclosure is configured and arranged to enable the delivery of an annuloplasty device. The annuloplasty device is configured and arranged to restore a heart valve annulus from a dysfunctional configuration to a working configuration. The annuloplasty device is configured and arranged to expand from a pre-set configuration corresponding to the working configuration of the valve annulus.

Within the context of the present disclosure, the annulus may have a “dysfunctional configuration” in which it has partially or completely lost its functional activity, namely, to provide structural support to the valve so that the valve allows flow from a first compartment to a second compartment and minimises backflow from the second compartment to the first compartment. A dysfunctional annulus may be (genetically or pathologically) degenerated and the terms are used herein interchangeably. The degeneration may for example originate from injury, malformation or injury, and often expresses itself as a loosening or dilatation of the annulus. By contrast, a “working configuration” is a configuration wherein the annulus provides adequate and sufficient structural support so as to minimise or avoid valve regurgitation.

Exemplary types of annuloplasty devices will be described hereinbelow, which can be used with or in the system and method of the present invention. The first exemplary annuloplasty device may be referred to as a “band-type” or “ring-type” annuloplasty device, interchangeably. The second exemplary annuloplasty device may be referred to as a “crown-type” annuloplasty device. The third exemplary annuloplasty device may be referred to as a “bridge-type” annuloplasty device.

According to a first example, the annuloplasty device may comprise a band which is expandable from a pre-set configuration, wherein the pre-set configuration of the band corresponds to the working configuration of the annulus.

The delivery system may exert a suitable external force to the annuloplasty device to expand from its pre-set working configuration to enable its anchoring to the dysfunctional annulus, and upon cessation of said force, the annuloplasty device automatically contracts or reverts back to its pre-set working configuration. This contraction mechanism is a key aspect of the annuloplasty device, in that it facilitates the simplification and standardisation of annuloplasty procedures. This is advantageous over previous transcatheter annuloplasty systems, which must be adjusted in situ, in the patient at the target site, by trained expert surgeons using remotely controlled tools and imaging equipment. By contrast, in the present system, minimal intervention, estimation and adjustment by the surgeon are required, either before (e.g. during the grasping and securing phase) or after release of the device.

The pre-set configuration is one which restores a dysfunctional annulus into a working annulus. While sizes may differ from patient to patient, the overall average three-dimensional shape of working heart annuli is similar in patients and commonly known. The pre-set configuration of the annuloplasty device may therefore translate into a three-dimensional shape suitable to restore the configuration of one or more sections of a dysfunctional annulus, and/or restore its overall configuration. The annuloplasty device is preferably manufactured with a pre-set configuration corresponding to the average three-dimensional shape of a working human heart valve for standardisation purposes, but may also be manufactured with modifications to treat specific defects and/or specific patient groups. The annuloplasty device may be supplied in different sizes.

Generally, “restoring” the annulus refers to the reduction of one or more dimensions of a pathological (large) annulus. The annuloplasty device and system seek to restore the annulus from a dysfunctional or degenerated configuration to a working configuration, and may also be used in the context of preventative and palliative procedures, where it is desired to maintain or preserve one or more dimensions or shape of the annulus.

As used herein, “expandable” or “expanded” means that one or more dimensions of the expanded annuloplasty device is greater than the one or more corresponding dimensions of the annuloplasty device in the pre-set configuration. The expanded band has a configuration such that it can be secured to the degenerated or dilated annulus.

As used herein, the term “means” can be equivalently expressed as, or substituted with, any of the following terms: device, apparatus, structure, part, sub-part, assembly, sub-assembly, machine, mechanism, article, medium, material, appliance, equipment, system, body or similar wording.

As used herein, “proximal” side refers to the side closest to the medical practitioner and outside the patient; “distal” refers to the side closest to the target annulus.

The present system does not require extensive manipulation and adjustment in situ. Once delivered to the target area, the annuloplasty device is positioned, and the band expanded to reach the attachment sites of the dilated annulus. Upon release, the expanded band collapses, contracts or reverts to its pre-set configuration, thereby automatically contracting the annulus into its working configuration to prevent significant amounts of blood flowing back through the heart valve.

The band may further be compressible from the pre-set configuration. As used herein, “compressible” or “compressed” means that one or more dimensions of the compressed annuloplasty device is smaller than the one or more corresponding dimensions of the annuloplasty device in the pre-set configuration. The band may be deformable, foldable or collapsible, so as to achieve an expanded configuration and/or a compressed configuration. The compressed band has a configuration such that it can be fitted in the inner lumen of a delivery catheter or delivery sheath. The compressed band may be slidably fitted in the inner lumen of a delivery catheter or delivery sheath.

Transcatheter procedures are minimally invasive and allow patients to experience less discomfort and to recover in a shorter amount of time, compared to surgical procedures. However, transcatheter procedures involve smaller devices and instruments, which are more difficult to manipulate, and the surgeon's movement is further limited by hindrance and obstacles in the patient's own anatomy. Access is not an issue with surgical procedures, and there is therefore little need for additional complex insertion, delivery, viewing and implantation instruments. The annuloplasty device may be both expandable and compressible, and requires minimal manipulation in situ.

The overall (or some) dimensions of the annulus in a working configuration are the same or smaller than the annulus in a degenerated configuration. Therefore, the annuloplasty device may have a pre-set configuration with overall (or some) dimensions which are smaller than the dimensions of the expanded device in the deployed configuration; and/or the annuloplasty device has a pre-set configuration with overall (or some) dimensions which are greater than the dimensions of the collapsed or compressed annuloplasty device in the delivery configuration.

The dimensions of the band in the expanded configuration are equal or greater than the dimensions of the degenerated annulus. Thus, the band can be expanded so as to reach the attachment sites of the annulus, and secure the band thereto.

The band, in its pre-set configuration, may be substantially rigid. The pre-set configuration of the band is a configuration which supports the annulus in a working configuration. Since the working configuration is the configuration in which the backwards flow is significantly minimised, there may be more than one working configuration of the annulus.

The band in its pre-set configuration may be sufficiently rigid to act as a support, scaffolding or skeleton for the annulus. When the band is secured in its pre-set configuration to the annulus, it does not only narrow the circumference of the dilated annulus, but it also re-shapes the annulus. The three-dimensional shape of the annulus is discussed in further detail hereinbelow.

The band in the pre-set configuration may substantially saddle-shaped. “saddle-shaped” includes bands comprising two or more curves with the apex facing a first direction, and two or more curves facing a second direction, the second direction being opposed to the first direction. The saddle shaped band may comprise two upward facing curves and two downwards facing curves. This pre-set configuration is particularly beneficial in that it defines the anatomical contours of a working annulus.

Other suitable shapes, contours or portions of the band allowing to reshape the annulus into a working configuration are envisaged including, but not limited to, an elongated member, a partial or complete ring, a partial or complete oval and the like. Angular shapes, contours or portions may be included, but curved or linear shapes are preferred to avoid tissue trauma and facilitating delivery of the device to the target site.

The band may be open or closed. The open band may be substantially linear, C-shaped, U-shaped, V-shaped, L-shaped and the like. The closed band may be substantially circular, oval, rectangular, D-shape and the like. Other shapes are envisaged, such as polygonal. The band may comprise one or more sections and each section may have the same or different shape.

The pre-set overall configuration of the band may be three-dimensional, as is the annulus.

The annuloplasty device, in particular the band, may comprise or consist of one or more plastics, rubber or metal materials. Said one or more plastics, rubber or metal materials may be elastic or have elastic properties. For example, the band may consist of one or more elastic materials so as to be expandable and/or compressible from its pre-set configuration. Alternatively, the band may comprise one or more sections consisting of one or more elastic materials.

Examples of materials include plastics such as silicone-containing materials, rubbers, fabrics, metals such as shape-memory materials and alloys (in particular nitinol).

The annuloplasty device may comprise or consist of one or more bioabsorbable materials. Bioabsorbable materials are particularly advantageous in that once the annuloplasty device is implanted, fibrous connective tissue will form around the annuloplasty device and will itself act as a natural support for the annulus. As the bioabsorbable materials are slowly absorbed, the annulus is maintained in its working configuration by the newly formed tissue.

The annuloplasty device may comprise a compressible and/or expandable structure. The compression and/or expansion may be achieved by mechanical means.

The band may comprise an expandable and/or compressible mesh or structure. For example, the band, mesh or structure may have a grid, diamond, serrated, crenelated, sinusoidal, spiral, helical and/or other patterns. The band may be integrally formed.

The band may comprise mechanical means such as hinged connections, elastic connections, springs, pivotable and or rotatable connections to achieve expansion and/or compression.

The annuloplasty device may comprise a tubular band, ring or crown comprising or consisting of any one or more of the materials, meshes or structures described above.

The outer surface of the band may comprise a protective cover, such as a layer of material and/or a coating. The cover may be provided to enhance the contact and interaction with the surrounding tissue, to promote fibrosis and tissue growth. The cover may comprise or consist of a mesh or a woven fabric.

The cover may comprise or consists of a flexible material, flexible enough to allow expansion and compression of the band. Examples of materials include polymers, such as polyester and polytetrafluoroethylene (PTF).

The protective cover may comprise one or more compounds to minimise the risk of thrombosis or inflammation. Examples of compounds include anti-coagulants, anti-inflammatory compounds, anti-bacterial compounds, anti-viral compounds, and the like. The protective cover may comprise one or more pro-healing compounds or compositions, i.e. compounds or compositions promoting healing.

The band could be anchored by means of sutures in a conventional manner. However, with the aim of providing an annuloplasty device, method and system which are simple and standardised, the annuloplasty device preferably comprises means for anchoring the band to the annulus.

The anchoring means may be mechanical anchoring means such as barbs, pegs, hooks, spikes and the like. The anchoring means may be either connected to, adhered to or glued to the band. Preferably integrally-formed with the band, to provide a safer device which is less resource intensive and simpler to manufacture. Using integrally-formed anchoring means also results in an annuloplasty device which is easier to implant, with fewer independently movable components.

Bioglues and bioadhesives are also envisaged within the context of the present disclosure, and the band, or its cover where applicable, may be coated with one or more adhesives instead of, or preferably, in addition to the mechanical anchoring means.

The annuloplasty device may comprise means for automatically deploying the anchoring means. The anchoring means may automatically deploy and/or secure to the annulus upon release of the band from its expanded configuration. The anchoring means may be connected to the band so that, in the pre-set configuration of the band, the anchoring means are in their anchored configuration.

The band may comprise anchoring means, which deploy and/or secure to the annulus by rotation of the band around its longitudinal axis (if the band is elongated) and/or by application of pressure (for example by pressing the anchoring means into the tissue of the annulus).

According to a second example, the annuloplasty device may comprise a support structure comprising a distal mouth. The support structure may be expandable from a pre-set configuration, wherein the pre-set configuration of the mouth corresponds to a working configuration of the annulus. The annuloplasty device may further comprise means for anchoring the mouth to the annulus.

In this second example, the band of the first exemplary annuloplasty device is replaced by the mouth of the expandable (and retractable) support structure, and the pre-set configuration of the mouth of the support structure corresponds to a working configuration of the annulus. In other words, the band and/or the mouth is the part of the annuloplasty device which surrounds and interacts with the annulus to support the annulus in its working configuration.

The mouth may be defined by the aperture of the annuloplasty device at a distal or proximal end thereof, for example by the outer periphery of the aperture.

The support structure fulfils the dual function of providing structural support to the annuloplasty device and of defining a mouth.

Structural support is advantageous in that it allows the mouth to return and remain in its pre-set configuration. In other words, it assists in the retraction of the mouth into the pre-set configuration during the implantation process and it minimises or prevents the deformation of the mouth once it is implanted on the annulus. The structural support may increase the inward force required to return the mouth to its pre-set configuration against the outward force exerted by the dilated annulus.

Structural support may for example be provided by mechanical means, the choice of material, shape and/or dimensions of the structural support. These features may be selected and adjusted to impart the intended support. For example, a material may be selected to provide the intended properties, including but not limited to expansion, retraction, resilience, rigidity and flexibility.

The structural structure may comprise a mesh structure, which may comprise a diamond-pattern. The diamond pattern is a simple structure allowing the control and adjustment of the expansion and retraction forces of the device, whilst providing structural rigidity to the device. The shape and dimensions of the diamonds and the pattern is not particularly limited. However, two rows have been shown to provide adequate support.

The structural support may comprise one or more resilient members, which may define substantially sinusoidal, crenelated or zigzag pattern. One or more members may be provided of the same or difference patterns.

The mouth has a pre-set configuration corresponding to a working configuration of the annulus, defined hereinabove in relation to the annuloplasty with a band. The mouth of the device may be defined by an aperture of the support structure (e.g. the distal aperture). The aperture may or may not comprise a band as described hereinabove in relation to the annuloplasty with a band.

Within the context of the present delivery system and method, it is envisaged to use an annuloplasty device according to the first example comprising a structural support as described in relation to the annuloplasty device according to the second example. In other words, the annuloplasty device may comprise a band and a structural support.

The annuloplasty device may comprise means for anchoring the mouth to the annulus. The anchoring means may be the same or different from the anchoring means described in relation to the annuloplasty with a band. Preferably, the anchoring means extend from the mouth of the support structure. The anchoring means may extend substantially longitudinally from the mouth of the support structure, or an angle inwards relative to the mouth of the support structure.

According to a third example, the annuloplasty device may comprise a bridge structure having a first end and a second end; first means for anchoring the annuloplasty device to the annulus at the first end of the bridge structure, and second means for anchoring the annuloplasty device to the annulus at the first end of the bridge structure; wherein the annuloplasty device is expandable from a pre-set configuration, wherein the pre-set configuration of the annuloplasty device is configured to support the annulus in its working configuration.

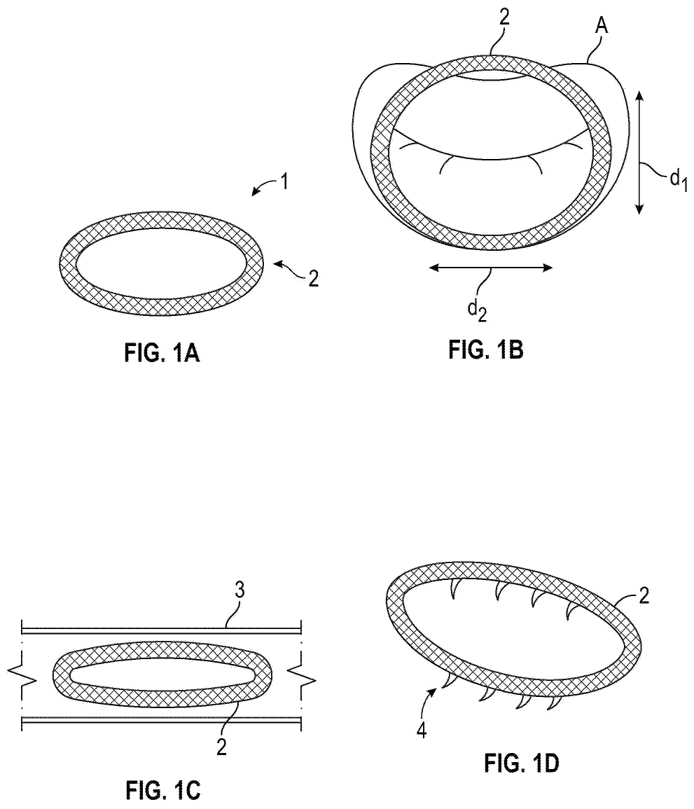

With reference to FIG. 1, there is illustrated an annuloplasty device 1 for restoring a heart valve annulus A from a dysfunctional configuration to a working configuration, wherein the device 1 comprises a band 2 which is expandable from a pre-set configuration, wherein the pre-set configuration of the band 2 corresponds to a working configuration of the annulus A.

In the example illustrated in FIG. 1, the annuloplasty device 1 comprises a closed band 2, which is tubular and formed of a mesh. The mesh structure is expandable as illustrated in FIG. 1B, and may be compressible as illustrated in FIG. 1C. The structure may have a pattern, such as a grid, diamond, serrated, crenelated, sinusoidal, spiral, helical and/or other patterns. Alternatively, the band 2 may be a solid band. The band 2 may comprise an elastic material or a shape-memory alloy, such as nitinol.

The band 2 is shown in an exemplary substantially oval pre-set configuration in FIG. 1A, which corresponds to a working configuration of the annulus A. In FIG. 1B, the band 2 is illustrated in an expanded anchoring configuration, in which the dimensions of the band 2 can be expanded in directions d1 and/or d2 to reach the attachment sites of the annulus A. In FIG. 1C, the band 2 is illustrated in a compressed delivery configuration, in which the band 2 is compressed into a delivery catheter 3, so that the band 2 can be pushed along the inner lumen of the delivery catheter 3. In FIG. 1D, the band 2 comprises exemplary anchoring means 4, in the form of pegs. The anchoring means 4 are connected to the band 2, partially or completely along the band 2.

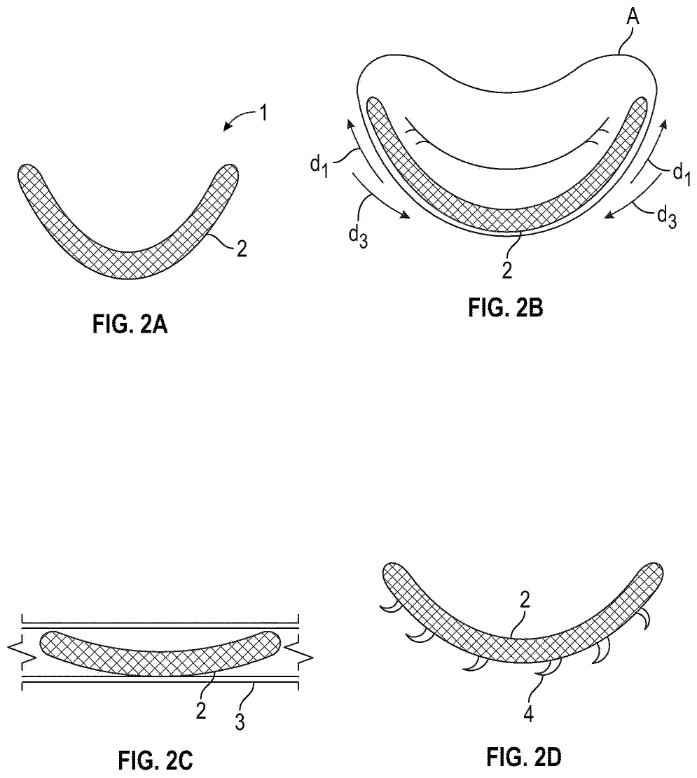

In the example illustrated in FIG. 2A, the annuloplasty device 1 comprises an open band 2, which is tubular and formed of a mesh. The band in FIG. 2A is shown in an exemplary curved pre-set configuration, which corresponds to a working configuration of the annulus A.

In FIG. 2a, the band 2 is illustrated in an expanded anchoring configuration, in which the dimensions of the band 2 can be expanded in directions d1. Upon release of the expanded band 2, the band 2 retracts into its pre-set configuration in direction d3.

In FIG. 2C, the band 2 is illustrated in a compressed delivery configuration, in which the band 2 is compressed into a delivery catheter 3, so that the band 2 can be pushed along the inner lumen of the delivery catheter 3. The band 2 may be compressed to that its radial dimension is decreased, or the band 2 may be folded upon itself to decrease its overall longitudinal dimension. In FIG. 2D, the band 2 comprises exemplary anchoring means 4, in the form of hooks. The anchoring means 4 are connected to the band 2, partially or completely along the band 2.

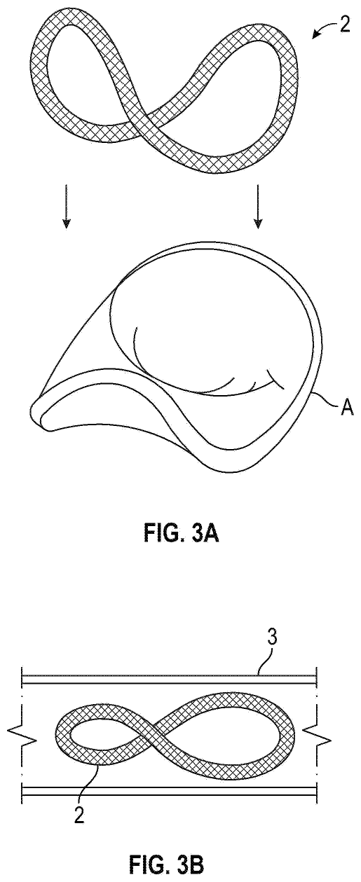

In the preferred example illustrated in FIG. 3A, the annuloplasty device 1 comprises a substantially saddle-shaped band 2. The shape of the band 2 in its pre-set configuration corresponds to and restores the annulus A to its working configuration. The band 2 may be expanded (not shown) in any direction owing to the material and/or the structure of the band 2, as described hereinabove. In the expanded configuration, the band 2 reaches and anchors to the attachment sites of the degenerated or dilated annulus A. Upon release of the band 2 from its expanded configuration, the band 2 reverts into its pre-set configuration, thereby reshaping the annulus A to its working configuration.

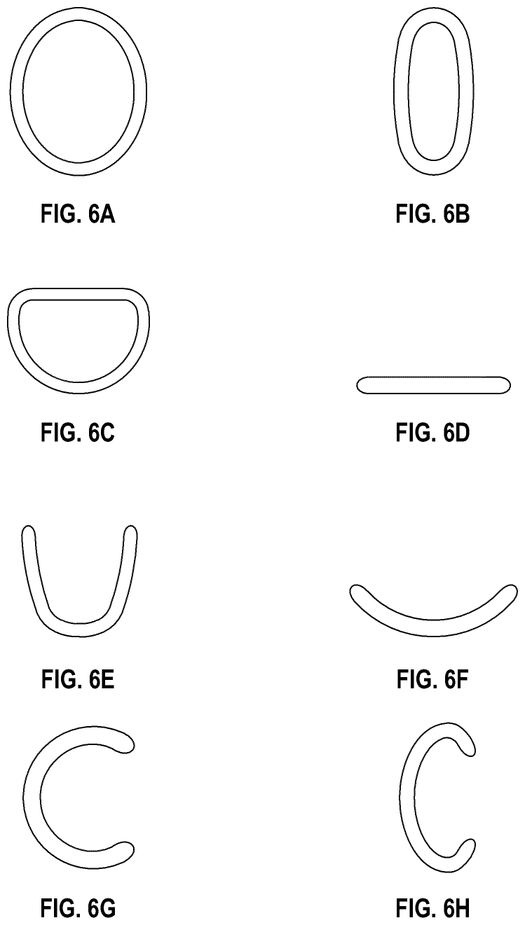

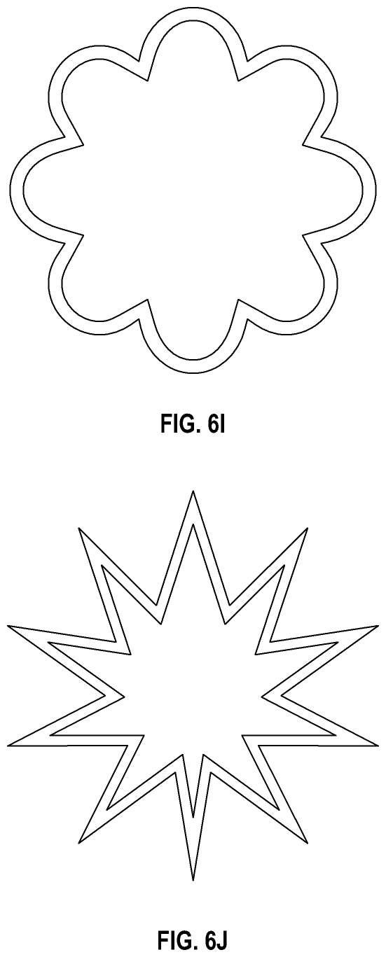

FIGS. 6a to 6h are top views of exemplary pre-set working configurations for the annuloplasty device 1. The device 1 is preferably three-dimensionally shaped to correspond to the three-dimensional shape of a working annulus and/or to restore a dysfunctional annulus to a working configuration. The device 1 may have a closed shape (as shown in FIGS. 61 to 6c) to surround the valve. Alternatively, the device 1 may have an opened shape (as shown in FIGS. 6d to 6h) to partially surround and restore the valve.

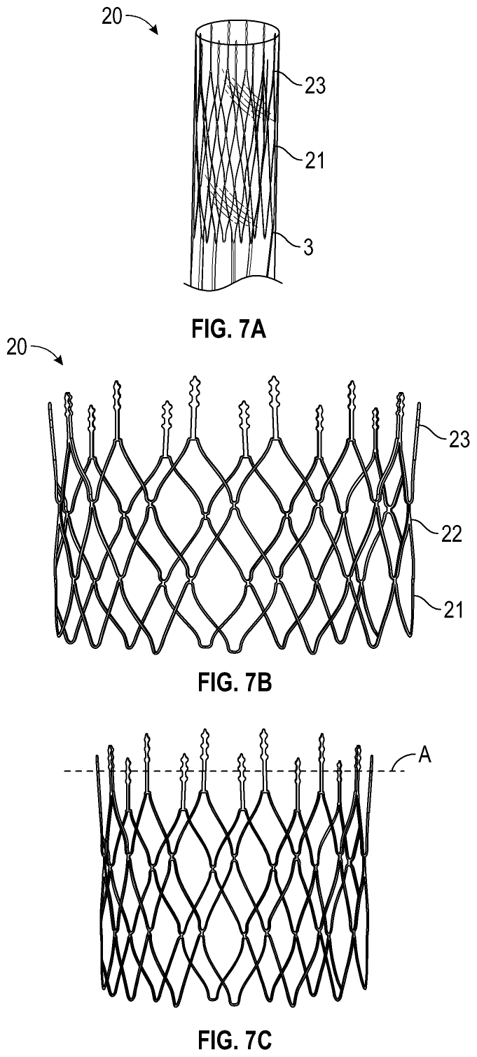

FIGS. 7a to 7c are provided to illustrate additional or alternative features of the annuloplasty device as described hereinabove (with reference to a “crown-type” annuloplasty device. An annuloplasty device 20 is provided with a support structure 21 comprising a distal mouth 22, wherein the support structure 21 is expandable from a pre-set configuration (FIG. 7b), wherein the pre-set configuration of the mouth 22 corresponds to a working configuration of the annulus; and means 23 for anchoring the mouth to the annulus.

In FIG. 7a, the annuloplasty device 20 is illustrated in its delivery configuration, folded so as to fit in a delivery catheter 3. In this embodiment, the annuloplasty device 20 is its folded configuration has smaller outer dimensions than those of the pre-set configuration.

In FIG. 7b, the annuloplasty device 20 is illustrated in its expanded configuration. During the implantation process, the support structure 21 is expanded (by means of a delivery device — not shown), so that the dimensions of the mouth 22 are greater than those of the annulus and greater than those of the mouth 22 in the pre-set configuration.

Once suitably positioned, the device 20 is released so that the anchoring means 23 (in the present embodiment comprising barbs) grab and anchor to or into the tissue surrounding the annulus. The mouth 22 reverts to its pre-set configuration as shown in FIG. 7c, so that the annulus is reverted to a working configuration.

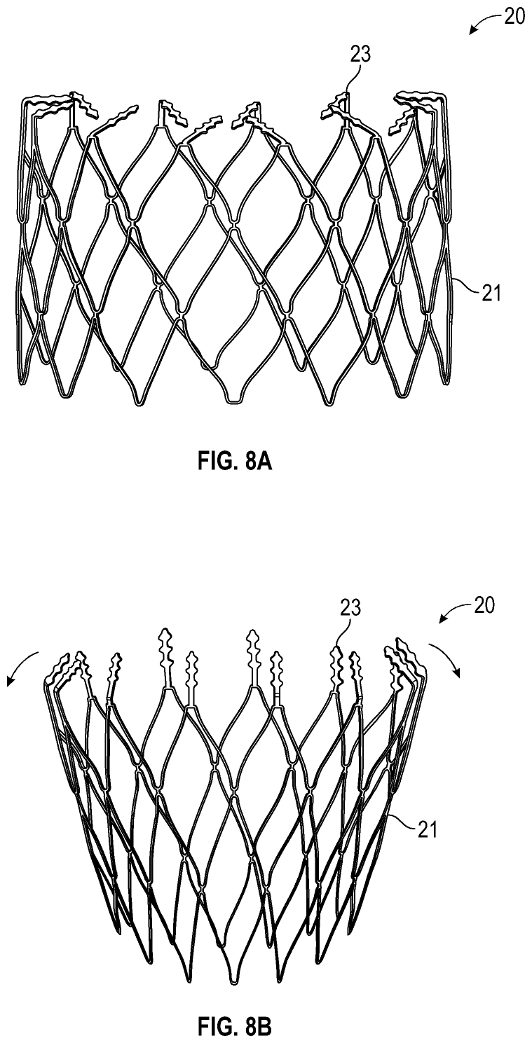

In FIGS. 7a to 7c, the anchoring means 23 extend longitudinally from the support structure 21. However, the anchoring means 23 may extend inwardly relative to the mouth and/or support structure as shown in FIGS. 8a and 8b. This inward angle has the advantage of improving the grabbing of and anchoring to the patient's tissues.

In situ, the support structure 21 may extend away from the annulus or may be located partially or completely through the annulus.

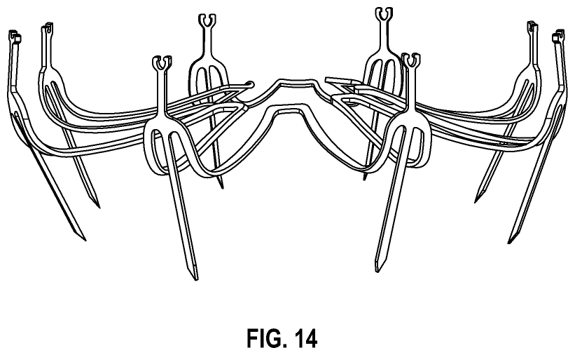

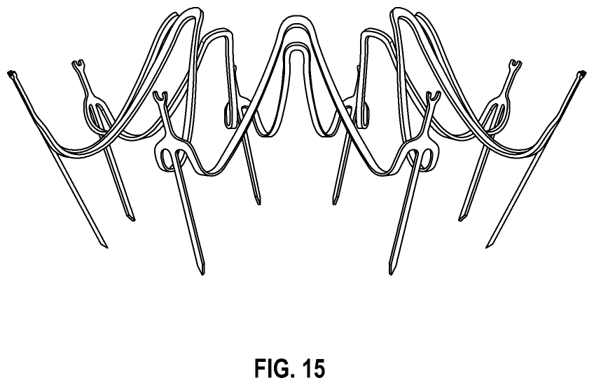

FIGS. 14 and 15 illustrate additional or alternative features of the annuloplasty device as described hereinabove with reference to another crown-type annuloplasty device 20 comprising anchoring means 23. In this embodiment, the annuloplasty device 20 may be integrally formed of a resilient material, such as a shape-memory material. The annuloplasty device 20 may also comprise attachment portions 40, which may extend longitudinally from, but towards the opposing direction to that of the anchoring means 23. The annuloplasty device 20 may comprise a lever portion, which may extend inwards from the main body (e.g. the crown portion) of the annuloplasty device 20. In the expanded configuration illustrated in FIG. 14, the lever portion pay be pushed downwards or in a distal direction, so that the anchoring means 23 expand outwards. In the pre-set configuration illustrated in FIG. 15, the lever portion is configured and arranged so as to not obstruct the mouth of the annuloplasty device. The annuloplasty device may comprise a reinforcing portion (illustrated as a handle shaped portion between the anchoring portion and the attachment portion) to provide rigidity and therefore stability to the annuloplasty device when the annuloplasty device is expanded.

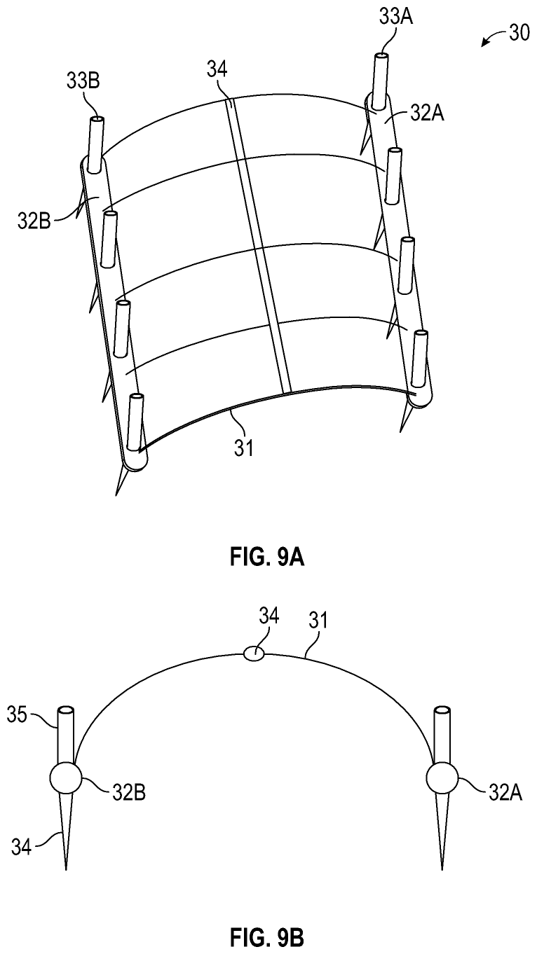

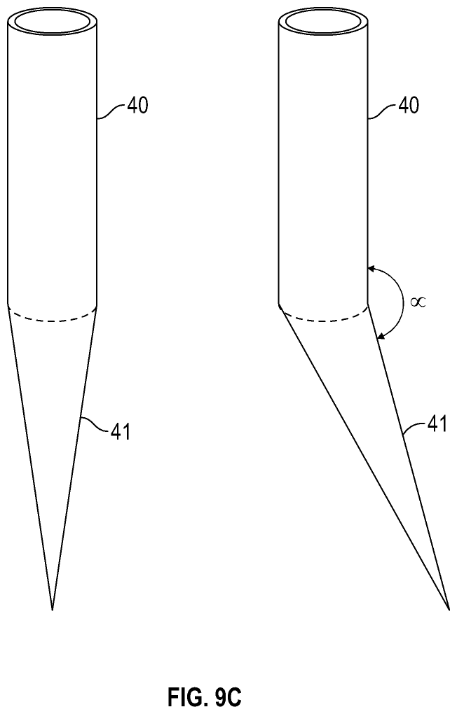

FIG. 9 is provided to illustrate additional or alternative features of the annuloplasty device as described hereinabove. An annuloplasty device 30 is provided with a bridge structure 31. The annuloplasty device 30 has a first band 32A and a second band 32B. The annuloplasty device 30 has first means 33A for anchoring the first band 32A to the annulus, and second means 33B for anchoring the second band 32B to the annulus, wherein the annuloplasty device 30 is expandable from a pre-set configuration, wherein the pre-set configuration of the annuloplasty device 30 is configured to support the annulus in its working configuration.

The bridge structure 31 may comprise or consist of a resilient material, such as a shape-memory material (e.g. nitinol), in the form or one or more bands or wires, or a mesh (which may be an expandable mesh structure as illustrated in relation to the “crown-type” annuloplasty device).

In some embodiments, the bridge may comprise a plurality of bands or wires connected to each other by means of a spine 34.

The annuloplasty device 30 may comprise a resilient bridge structure 31 comprising at least one anchor at each end thereof. The annuloplasty device 30 may be folded for delivery through a sheath. The annuloplasty device 30 may deploy upon exiting the sheath. The annuloplasty device 30 may be expanded or stretched beyond its pre-set configuration. The annuloplasty device 30 may be anchored to the annulus by means of the anchoring means, and released into its pre-set configuration.

The anchoring means 33A, 33B may be the same or different from the anchoring means described in relation to previous annuloplasty devices. In a preferred embodiment, the anchoring means 33A, 33B comprises a pin portion 34 to anchor into the patient's tissues, and an attachment portion 35 to attach the annuloplasty device to the delivery device. The pin portion 34 and the attachment portion 35 may be integrally formed. The anchoring means 33A, 33B may be integrally formed with its corresponding bands 32A, 33A, or may be mechanically coupled thereto.

FIG. 9C illustrates a preferred anchoring means for use in the context of the present disclosure, and which may be used with any annuloplasty device as described herein.

The anchoring means comprises an attachment portion 40 to detachably couple the annuloplasty device to the delivery device. The attachment portion 40 may comprise a substantially elongated member and may be have a circular cross-section (although other cross-sectional configurations are envisaged).

The anchoring means comprises an anchoring portion 41 intended to penetrate the patient's tissues. The anchoring portion 41 may be substantially be pin shaped, e.g. may comprise a substantially tapered (sharp) end. The pin shape is preferred for example to barbs or screws because, during the anchoring process, anchoring portions comprising barbs or screws tend to dig a hole about themselves when entering the patient's tissues so that they tend to be less securely retained. By contrast, pins are less traumatic and are retained due to the inward force the of the expandable annuloplasty device.

The attachment portion 40 and the anchoring portion 41 may be integrally formed, or may be mechanically coupled to each other. In the preferred embodiment, the attachment portion 40 and the anchoring portion 41 are integrally formed. The attachment portion 40 and the anchoring portion 41 may form an angle a of 135° or 180°. The angle a may be comprised between 90° and 180°, preferably between 135° and 180°. The value of the angle a depends on the amount of expansion required to provide optimal entry into the tissue and optimum grasping of the tissue.

Within the context of the present disclosure, the anchoring means may be configured in the pre-set configuration so that they extend perpendicularly, inwards or outwards from the main body of the annuloplasty device (e.g. band, ring, crown, and/or supporting portion). The anchoring means may be positioned in a symmetrical, concentric, asymmetrical or non-concentric manner along the main body of the annuloplasty device.

The annuloplasty device may be used to minimise or prevent further deterioration of the annulus', for improving or restoring the annulus' function and/or for palliative support. The device is simple and standardisable. The development and implantation of the device in patients becomes less resource-intensive when compared to conventional devices. The present annuloplasty device can be delivered and implanted using transcatheter systems and methods according to the present disclosure, which are minimally invasive. The annuloplasty device is easily secured to a dysfunctional annulus in order to restore it to a healthy working configuration. As will be described hereinbelow, the annuloplasty device can be delivered expediently and accurately using the systems and methods according to the present disclosure, which require minimal manipulation, positioning and adjustment in situ. The medical practitioners require less extensive training as would be required to implant conventional annuloplasty devices. Importantly, the annuloplasty device restores the shape, in particular the three-dimensional shape, of the annulus instead of simply reducing the opening of the valve.

### Delivery System

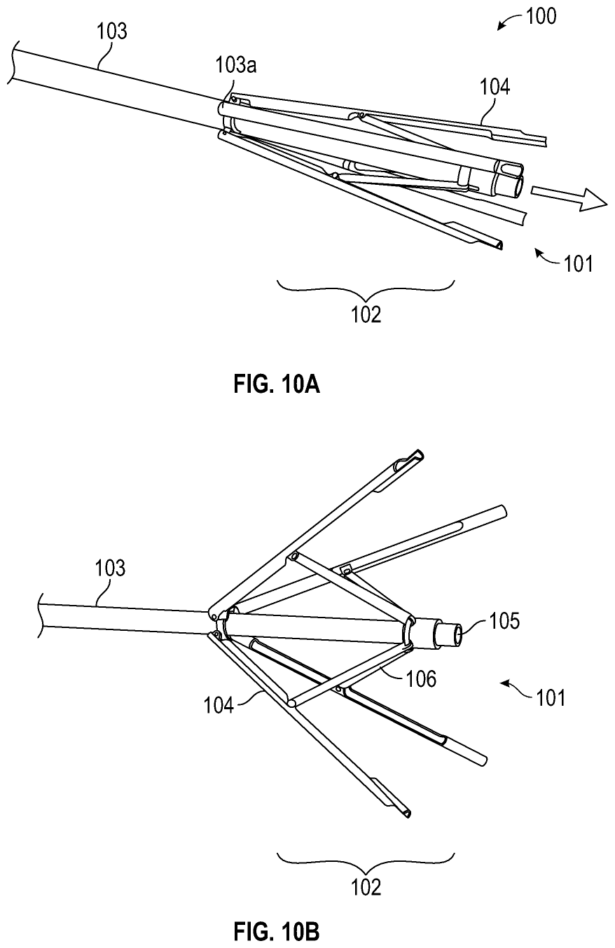

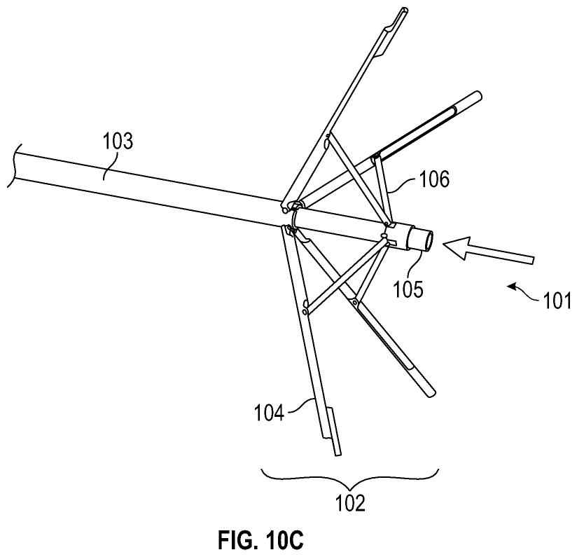

With reference to FIGS. 10, there is illustrated a system 100 for the delivery of an annuloplasty device 1,20,30 for restoring a heart valve annulus from a dysfunctional configuration to a working configuration, wherein the annuloplasty device 1,20,30 is configured to expand from a pre-set configuration corresponding to the working configuration of the valve annulus, the system 100 comprising a delivery device 101 configured to be detachably coupled to the annuloplasty device 1,20,30, wherein the delivery device 101 comprises a structure 102 configured to expand from a first configuration to a second configuration, wherein the dimensions of the second configuration are the substantially the same or greater than the dimensions of the pre-set configuration of the annuloplasty device 1,20,30.

In the embodiment illustrated in FIGS. 10A, 10B, 10C, the delivery system 100 comprises a delivery device 101 comprising an expandable structure 102 positioned at or adjacent the distal end 103a of a shaft 103.

The expandable structure 102 comprises a plurality of arms 104. Each arm 103 comprises a proximal end and a distal end. In FIGS. 10A, 10B, 10C, the proximal ends of the arms 104 are coupled to the shaft 103 (e.g. adjacent or at the distal end 103 of the shaft 103). The proximal ends of the arms 104 are pivotably coupled to the shaft 103 and are configured and arranged to pivot about distal end 103a (e.g. about the point where the proximal end of the arm 104 connects with the shaft 103).

The arms 104 in FIG. 10A are shown in a first substantially folded configuration, suitable to fit the delivery device 101 through a delivery sheath (not shown).

In FIG. 10B, the arms 104 are shown in a second configuration wherein the dimensions of the second configuration are substantially the same as the dimensions of the pre-set configuration of the annuloplasty device. In other words, the expandable structure (more particularly, the distal ends of the arms 104) defines a space, the dimensions of which are substantially the same as the dimensions of the pre-set configuration of the annuloplasty device 1,20,30.

In FIG. 10C, the arms 104 are shown in a second configuration wherein the dimensions of the second configuration are greater than the dimensions of the pre-set configuration of the annuloplasty device. In other words, the expandable structure (more particularly, the distal ends of the arms 104) defines a space, the dimensions of which are greater than the dimensions of the pre-set configuration of the annuloplasty device 1,20,30.

The delivery device 102 comprises an actuation rod 105 and a plurality of stretchers 106. The actuation rod 105 is slidably received in a lumen of shaft 103, and the distal end of the actuation rod 105 extends beyond the distal opening of the lumen of the shaft 103.

Each stretcher 106 have a proximal and a distal end. In this example, the distal end of the stretcher 106 is pivotably coupled to the actuation rod 105, adjacent the distal end of the actuation rod 105. The distal end of the stretcher 106 may be directedly coupled to the actuation rod 105 or may be coupled to a runner or connector affixed to the actuation rod 105. The proximal end of the stretcher 106 is coupled to one or more arms 104. Each arm 104 comprises a proximal end, a distal end and an intermediary portion extending therebetween. In this example, the proximal end of the stretcher 106 is coupled to the intermediary portion of the arm 104, adjacent the mid-point of the arm 104.

Alternative or additional expansion mechanisms are envisaged, involving for example a balloon positioned between the arms 104, which may be selectively inflated and deflated to expand or fold the arms 104, or involving resilient or shape-memory arms 104 configured and arranged to automatically deploy upon exiting the delivery sheath. In the latter embodiment, a retraction mechanism is required to fold the arms and/or release the annuloplasty device. It is however noted that balloon expansion mechanisms are less preferred in that they tend to block blood flow through the annulus during the delivery process.

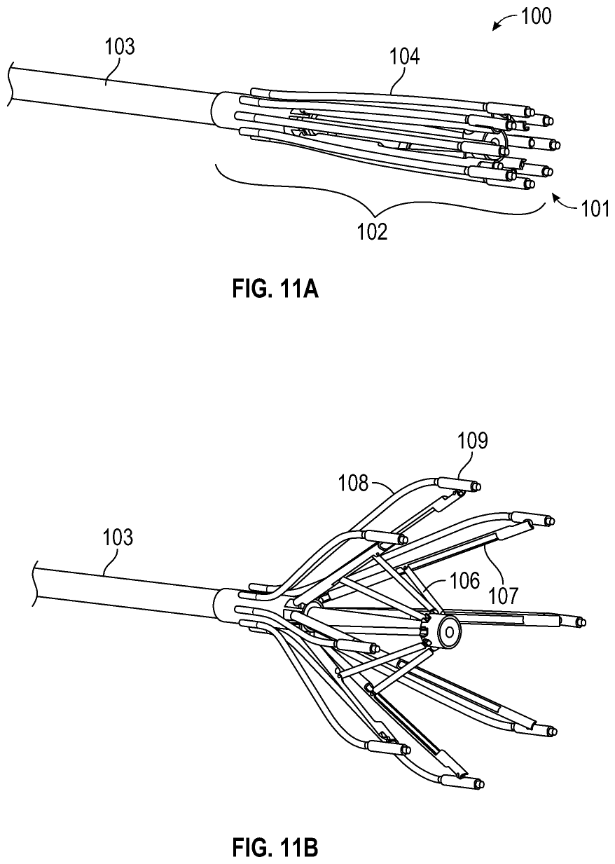

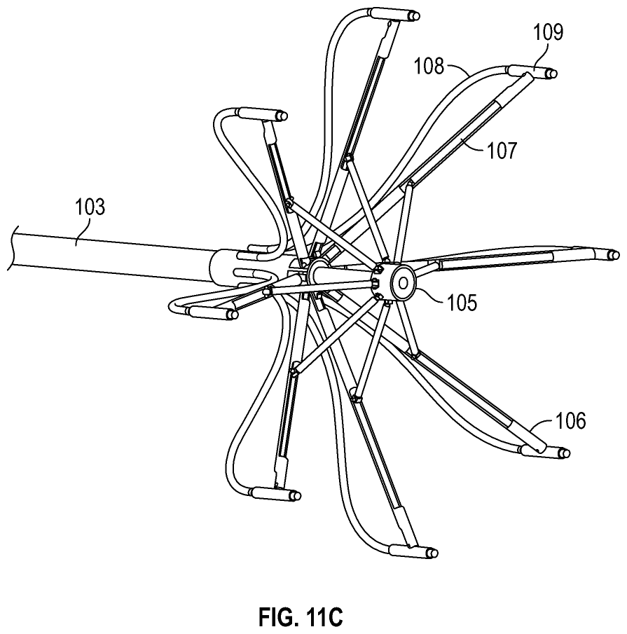

In the embodiment illustrated in FIGS. 11A, 11B, 11C, the delivery system 100 may comprise a delivery device 101 comprising an expandable structure 102, a shaft 103, arms 104, an actuation rod 105 and stretchers 106 as described in the embodiments of FIGS. 10A, 10B,10C.

The arms 104 may comprise grooves 107 configured and arranged to receive stretchers 10, in particular when the expandable structure 102 is in a folded configuration as illustrated in FIG. 11A.

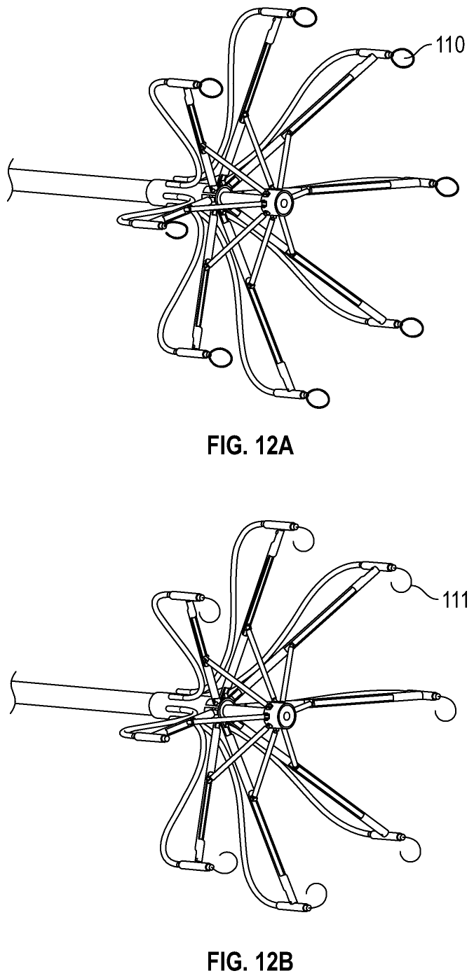

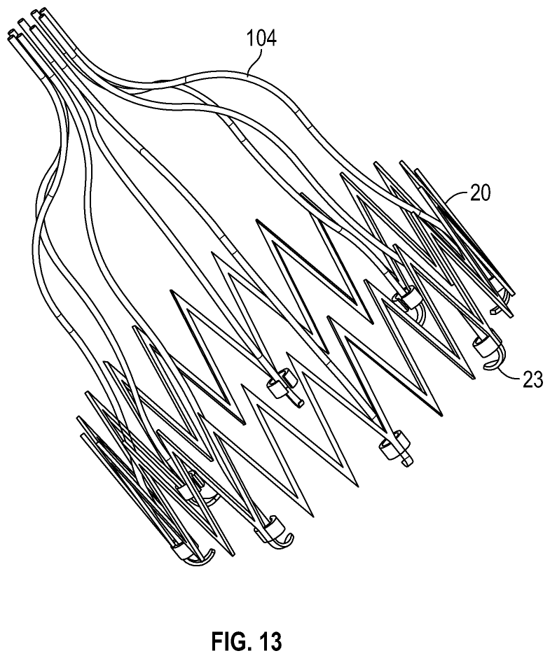

The delivery device 101 comprises one or more connectors for detachably coupling the expandable structure 102 to the annuloplasty device 1,20,30. These connectors may comprise a detachable loop connector 110 or hook connector 111, as illustrated in FIGS. 12A and 1C, respectively.

The connector structure comprises a plurality of ribs 108. The ribs 108 comprise a proximal end and a distal end, and in this example, the proximal end of the rib 108 is coupled to the shaft 103. The distal end of the rib 108 may be coupled to the distal end of the arm 104, either directly or via a connector outlet 109.

The ribs 108 are configured and arranged so as not to hinder the movement of the arms 104. For example, the ribs 108 may comprise or consist of a flexible material.

One or more ribs 108 may comprise a channel (not shown) to house a connector control line (not shown). The channel and connector control line may extend from the proximal user side to the connector outlet 109.

The connector outlet 109 may comprise a lumen (not shown) configured and arranged to receive an anchoring means, or more specifically to receive the attachment portion 40 of an anchoring means.

The lumen of the connector outlet 109 may have dimensions (slightly) greater than the outer dimensions of the anchoring means or of the attachment portion 40 thereof. In this configuration, the attachment portion 40 is retained in the lumen owing to the relative dimensions of the lumen and of the lumen of the connector outlet 109. The attachment portion 40 may be detached from the connector outlet 109 owing to the relative dimensions of the lumen and of the lumen of the connector outlet 109. In other words, the anchoring means or its the attachment portion 40 is retained (by relative configuration or by friction) due to the inner dimensions of the lumen being only slightly larger than those of the anchoring means or its the attachment portion 40. Other retaining means are envisaged, including but not limited to tabs, clips, screws, hooks and the like. The delivery device may comprise means for pushing the anchoring means out of the lumen of the connector outlet 109.

The connector outlet 109 may be configured to allow the passage of for example a hook connector 111 or loop connector 110 therethrough. The connector outlet 109 may be movable, for example pivotable, relative to the arm 104. The connector outlet 109 may comprise a substantially flat surface or a substantially curved surface to prevent the distal end of the arm 104 from injuring the patient.

The delivery device 100 may comprise guiding rails (not shown) extending from the arms 104. The guiding rails may be coupled of couplable to the arms 104. A guiding rail may be slidable along an arm 104 or received within an arm 104 so as to be extendable beyond the distal end of the arm 104.

Within the context of the present disclosure, there is also provided a control device for a delivery device as described herein. The control device comprises a control unit and a catheter extending therefrom. The catheter may be configured and arranged so as to receive the delivery device 100 and the annuloplasty device.

The catheter of the control device may comprise a support rod (preferably made of a relatively rigid material) to facilitate the alignment and positioning of the delivery device, and hence to improve the alignment and positioning of the annuloplasty device. The catheter of the control device may comprise a bend or curvature in order to facilitate steerability.

The control unit may comprise means controlling the rotation, extension and/or retraction of the delivery device, means for controlling the folding and/or expanding the delivery device, means for controlling the extension and/or retraction of the guiding rails, means for controlling the release of the annuloplasty device, and the like.

### Delivery Method

The present disclosure also concerns methods for restoring a heart valve annulus, comprising the step of delivering an annuloplasty device for restoring a heart valve annulus from a dysfunctional configuration to a working configuration, wherein the annuloplasty device is configured to expand from a pre-set configuration corresponding to the working configuration of the valve annulus, the system comprising a delivery device configured to be detachably coupled to the annuloplasty device, using a delivery system as described herein.

The methods may include any one or more of the following steps:
The step of coupling the annuloplasty device to the expandable structure of the delivery device.
The step of compressing the annuloplasty device, by means of the delivery device, to slidably fit in the lumen of a delivery catheter or sheath, so as to deliver the device to the target area.
The step of inserting the delivery system into a delivery sheath, wherein the expandable structure is in a folded configuration.
The steps of expanding the expandable structure into the second configuration.
The step expanding or deforming the annuloplasty device to a dimension equal or greater than a dimension of the heart valve annulus, so as to contact the anchoring sites on the dilated annulus.
The step of anchoring the annuloplasty device by releasing the expanded band into its pre-set configuration.
The step of releasing the annuloplasty device into its pre-set configuration.

Preferably, the heart valve is the mitral valve, and the annulus is the anterior annulus, the posterior annulus, the lateral commissure and/or the medial commissure. Most annuloplasty procedures are directed at reducing the posterior annulus. However, the portions between the anterior and posterior annulus (the commissures) are also known to elongate over time and can be reshaped using an annuloplasty device as described in. Preferably, the heart valve is the tricuspid valve, and/or the annulus is the anterior annulus, the posterior annulus and/or the septal annulus.

Within the context of the present disclosure, the annuloplasty device, the delivery device and corresponding methods may be used for the restoration of the aortic valve or the pulmonary valve.

Within the context of the present disclosure, the annuloplasty device, the delivery device and corresponding methods may be used for the restoration of the annulus, including any structural degeneration through and adjacent the annulus. For example, the annuloplasty device, the delivery device and corresponding methods may be used in the context of a paravascular leak (PVL).

A method for repairing a heart valve will now be outlined, using an annuloplasty device 1 as illustrated in FIG. 3A. It is noted here that the methods and systems according to the present invention may be used with any annuloplasty device as described herein.

The annuloplasty device 1 is detachably connected to a delivery system 100.

In a preferred embodiment, the annuloplasty device comprises anchoring means with an attachment portion 40. The attachment portions 40 are slid into the lumen of a connector outlet 109 (or directly into a lumen of the arm 104 for example if the delivery device does not comprise a connector outlet 109).

Additionally or alternatively, the loop connectors 110 may be coupled to the annuloplasty device 1 so as to surround the diameter of the band(s) or ring 2. Or, the hook connectors 111 may be coupled to the annuloplasty device 1 so as to partially surround the diameter of the band or ring 2. The annuloplasty device 1 may be coupled to the delivery system 1 whilst the expandable structure 102 is in the second configuration, for example as shown in FIG. 10B.

Other types of annuloplasty devices, such as “crown-type” annuloplasty device 20 and “bridge-type” annuloplasty device 30 may be coupled to the delivery device 101 is a similar manner.

The actuation rod 105 of the delivery device 101 is extended, in the proximal to distal direction. The expandable structure 102 is folded as illustrated for example in FIGS. 10A and 11A, thereby compressing the annuloplasty device in a folded configuration. The annuloplasty device 1 connected to the delivery device 101 is ready to be inserted into a delivery catheter 3. The delivery catheter 3 may be connected to a control device (not shown).

When the distal end of the catheter is adjacent the annulus, the annuloplasty device 1 is pushed by the delivery device along the length of the catheter 3. The distal end of the delivery catheter 3 is positioned adjacent the pathologically degenerated annulus A.

As the device 1 exits the delivery catheter 3, the annuloplasty device 1 may naturally reverts to its pre-set configuration. Additionally or alternatively, the actuation rod 105 of the delivery device 101 is retracted through a lumen of the shaft 103, in the distal to proximal direction, thereby expanding the expandable structure 102.

The proximal ends of the arms 104 are pivotably connected to the shaft 103. The proximal end of the stretchers 106 are pivotably connected to the shaft 103. Upon retracting the actuation rod 105, the arms 104 and the stretchers 106 are pushed against each other, so that the arms 104 and the stretchers 106 expand radially outwards and away from the actuation rod 106.

The expanded structure 104 is expanded into its second configuration, the dimensions of the second configuration are greater than the dimensions of the pre-set configuration of the annuloplasty device 1. That is, the expanded structure 104, and for example the distal ends of the arms 104, define a space with dimensions greater than the dimensions of the pre-set configuration of the annuloplasty device 1, so as to expand the annuloplasty device 1 from its pre-set configuration.

The annuloplasty device 1 is positioned so that its contours correspond to the general contours of the annulus (as shown in FIG. 3A). Finer adjustments may be made for example by expanding one or more arms 104 of the delivery device 101.

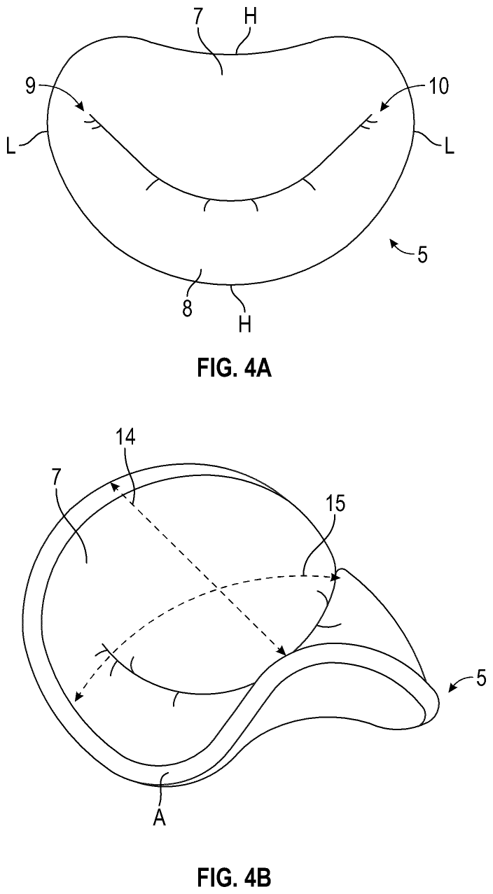

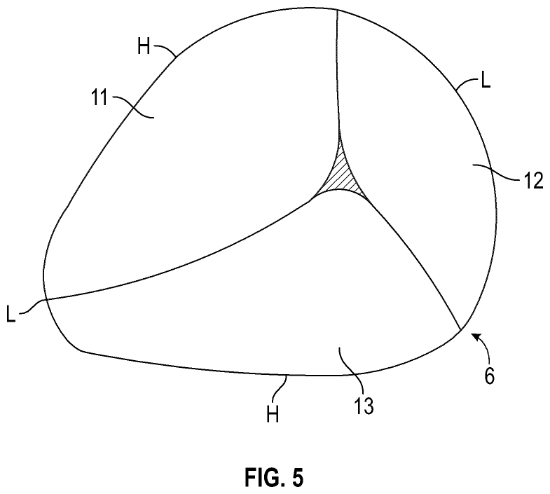

For a better understanding of the present invention, reference is made to FIGS. 4a, 4b and 5, illustrating the anatomy of the mitral valve 5 and the tricuspid valve 6.

The mitral valve 5 comprises an anterior leaflet 7, a posterior leaflet 8, a lateral leaflet 9 and a medial leaflet 10. The tricuspid valve 6 comprises an anterior leaflet 11, a posterior leaflet 12 and a septal leaflet 13.

The mitral valve 5 and tricuspid valve 6 do not have a planar configuration, but are curved and saddle-shaped. FIGS. 4a and 5 show the relatively high areas H and the relatively low areas L. The annuloplasty device 1 preferably does not have a substantially planar configuration. Instead, the device 1 is curved, and preferably saddle-shaped, so as to follow the natural configuration of the annulus A.

The device 1 is positioned adjacent the annulus A so that the device 1 is above the area of the annulus to be reshaped. This area may be the whole perimeter of the annulus A, or one or more portions of the annulus A. For example, a saddle-shaped device 1 may be used to reshape the whole annulus, or an open band device 1 may be used to reshape e.g. the posterior leaflet 8, 12 only.

From this position, and using the delivery device 101, the annuloplasty device 1 is expanded from its pre-set configuration so that the anchoring means 4 reach the attachment sites of the degenerated annulus. The attachment sites are preferably such that the configuration they outline has greater dimensions than those of the pre-set configuration of the annuloplasty device 1. Therefore, in order to grasp and anchor to the attachment sites of the annulus A, the annuloplasty device 1 is expanded.

If the delivery device 101 comprises guiding rails, these may be extended beyond the distal end of the arms 104 until the distal end of the guiding rails contact the patient's tissue. Thus, the user may assess the anchoring site of the annuloplasty device 1, before releasing the annuloplasty device 1.

Once the annuloplasty device 1 is suitably positioned, it may be released. This can be achieved for example by pushing or pulling the anchoring means out of the connection outlets 109.

If the delivery device 101 comprises guiding rails, the annuloplasty device 1 may slide along the guiding rails to reach the target anchoring position.

The anchoring means 4 are pressed into and secured to the tissue of the annulus A upon release of the annuloplasty device 1, owing to the annuloplasty device 1 reverting to its pre-set configuration. Alternatively or additionally, the delivery device 101 may be used to press on the anchoring means 4, and/or the arms 104 may be used to manipulate and/or rotate the band 2 so that the anchoring means 4 (for example hooks) grasp and secure to the annulus A.

Once the annuloplasty device 1 is suitably positioned and expanded, the anchoring means 4 are anchored to the attachment sites of the annulus A. The annuloplasty device 1 may be sutured to the attachment sites. However, it is preferred to avoid such an intricate and irreversible procedure by providing the annuloplasty device 1 with anchoring means. The anchoring means 4 may be hooks or pegs or other means which may be pushed into the tissue of the annulus A.

It is also envisaged that the annuloplasty device 1 is anchored to the “back” of the annulus, for example on the ventricular side of the annulus. In this embodiment, the delivery sheath 3 is pushed through the annulus, until the distal end of the sheath 3 is positioned beyond the annulus. The annuloplasty device 1 is pushed forward, and deploys upon exiting the delivery sheath 3. The annuloplasty device 1 is expanded beyond its pre-set configuration and suitably positioned to catch the annulus. The annuloplasty device 1 is anchored to the back of the annulus and released into its pre-set configuration.

It may be advantageous to provide structural support to the back of the annulus, if the back of the annulus is better defined, has better tissue integrity and provides more suitable anchoring sites. In addition, the anchoring step may be improved by using the pulling (in the proximal to distal direction) the annuloplasty device 1 to provide greater anchoring force.

A problem arising from conventional annuloplasty rings is that their implantation is difficult to reverse. In other words, the rings cannot be easily removed and repositioned in the event of an unsuccessful implantation. In the present invention, anchoring means 4 secured to the annulus by pressure from the annuloplasty device 1 (owing to its pre-configuration) may be used, so that should the annuloplasty device 1 require repositioning, the expansion of the device 1 would release the anchoring means 4. The annuloplasty device 1 can be repositioned with minimal trauma to the annulus A.

Also, when the annuloplasty device 1 is first released, the annuloplasty device 1 is still coupled to the delivery device 101 by means of the loops 110 or hooks 111. If the annuloplasty device 1 is not correctly positioned, it may be retrieved by pulling the loops 110 back, with the annuloplasty device 1 coupled to them.

Once the annuloplasty device 1 is securely anchored to the annulus, it may be detached from the delivery device 101. Where the annuloplasty device 1 is connected by means of loop connectors 110, the connectors 110 may be detached by pulling on one end of the connector line to free the annuloplasty device 1. Alternatively, the loop may be severed simply by pulling on the connector line to break the loop and free the annuloplasty device 1, or by other mechanical or chemical means. Where the annuloplasty device 111 connected by means of resilient or shape-memory hook connectors 111, the connector line may be pulled so as to retract the hooks 111 through the connector outlets 109.

The expandable structure 102 may be folded by extending the actuation rod 105, in a proximal to distal direction. The delivery device 101 may be retracted into the delivery catheter 3.

Although the present invention has been described within the context of heart valves and in particular of annuloplasty procedures, it is envisaged that it could have other advantageous implementations involving the reshaping of an anatomical structure.

The present invention is particularly beneficial in the context of transcatheter procedures and/or in the cardiovascular field. For example, in transcatheter procedures, access points are formed through tissues and/or blood vessels. The annuloplasty device may be used to restore blood vessels to their working configuration by partially or completely closing the access point(s) so as to minimise or prevent blood flow to the outside of the blood vessels.

The present invention may be used in the context of an atrial septal defect (ASD), which is a hole in the atrial septum. The ASD may be congenital, or may be the desired result of a medical procedure intended to relieve the pressure between the two atrial chambers of the heart. The annuloplasty device may be used to partially or completely close the hole so as to minimise or prevent blood flow between the atrial chambers.

Importantly, the present invention provides systems and methods specifically adapted to the expedient delivery and accurate positioning of the annuloplasty device.

1. A system for the delivery of an annuloplasty device for restoring a heart valve annulus from a dysfunctional configuration to a working configuration, wherein the annuloplasty device is configured to expand from a pre-set configuration corresponding to the working configuration of the valve annulus, the system comprising:
a delivery device configured to be detachably coupled to the annuloplasty device, wherein the delivery device comprises an expandable structure configured to expand from a first configuration to a second configuration, wherein dimensions of the second configuration are substantially the same or greater than dimensions of the pre-set configuration of the annuloplasty device.

2. The system according to claim 1, wherein the expandable structure comprises a shaft and one or more arms, each arm being movably connected to said shaft.

3. The system according to claim 2, wherein the arms are configured to fold radially inwards into the first configuration.

4. The system according to claim 2, wherein the arms are configured to expand radially outwards into the second configuration.

5. The system according to any one of claims 2, comprising an actuator configured to control the expansion of the expandable structure.

6. The system according to claim 5, wherein the actuator comprises an actuating rod which is slidable relative to the longitudinal axis of the shaft, and a plurality of stretchers coupled to the arms and the actuating rod.

7. The system according to claim 6, wherein the shaft comprises one or more lumens, the actuating rod being housed in at least one of the lumens of the shaft.

8. The system according to claim 5, wherein the actuator comprises an actuating sleeve which is slidable relative to the longitudinal axis of the shaft, and a plurality of stretchers coupled to the arms and the actuating sleeve.

9. The system according to claim 8, wherein the actuating sleeve comprises one or more lumens, the shaft being housed in at least one of the lumens of the actuating sleeve.

10. The system according to claim 2, wherein the delivery device includes one or more connectors for detachably coupling the expandable structure to the annuloplasty device.

11. The system according to claim 10, wherein the connector comprises a detachable loop connector or hook connector.

12. The system according to claim 11, wherein the connector comprises one or more ribs configured and arranged to receive a connector control line.

13. The system according to claim 12, wherein the expandable structure comprises a shaft and one or more arms movably connected to said shaft, and wherein the rib comprises a distal end coupled to the distal end, or adjacent the distal end of an arm.

14. The system according to claim 11, wherein the expandable structure comprises a shaft and one or more arms movably connected to said shaft, wherein one or more arms are configured and arranged to receive a connector control line.

15. The system according to claim 13, wherein the rib or arm comprises a connector outlet, said connector outlet being pivotably coupled to the rib or arm.

16. The system according to claim 2, further comprising a control device including a control unit and a catheter extending therefrom.

17. The system according to claim 16, wherein the catheter comprises a support rod.

18. The system according to claim 1, further comprising the annuloplasty device.

19. A method for restoring a heart valve annulus, comprising:
delivering an annuloplasty device for restoring the heart valve annulus from a dysfunctional configuration to a working configuration with a delivery device coupled to the annuloplasty device;
expanding the annuloplasty device from a first pre-set configuration to the working configuration of the valve annulus;
decoupling the delivery device from the annuloplasty device.

20. The method according to claim 19, comprising coupling the annuloplasty device to an expandable structure of the delivery device.

21. The method of claim 20, comprising inserting the delivery system into a delivery sheath, wherein the expandable structure is in a folded configuration.

22. The method of any one of claims 21, comprising expanding the expandable structure into a second configuration that corresponds to the working configuration.

23. The method of claims 22, comprising releasing the annuloplasty device into the pre-set configuration.

24. (canceled)
